(19) United States
(12) Patent Application Publication
(10) Pub. No.: US 2004/0072200 A1
(43) Pub. Date: Apr. 15, 2004

(54) Detection of nucleic acid polymorphisms

(76) Inventors: Rudolf Rigler, St-Sulpice (CH); Lars Edman, Stockholm (CH); Zeno Foeldes-Papp, Graz (AT)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/416,574

(22) PCT Filed: Nov. 13, 2001
(86) PCT No.: PCT/EP01/13120

(30) Foreign Application Priority Data
Nov. 13, 2000 (DE) 100 56 226.4
Dec. 29, 2000 (DE) 100 65 631.5

(51) Int. Cl. C12Q 1/68

(52) U.S. Cl. 435/6

(57) ABSTRACT

Methods are described methods functioning at the single-molecule level for characterizing nucleotide polymorphisms.

### DESCRIPTION

The present invention relates to a method for detecting single or multiple nucleic acid polymorphisms by detection of single fluorescence-labeled deoxyribonucleic acid molecules.

The genomes of the individuals of a species differ in sequence owing to nucleic acid insertions and deletions, differences in the number of repeats of short, recurring sequence motifs (so-called microsatellites and minisatellites) and differences in single base pairs, which-are referred to as single nucleotide polymorphisms (SNPs) and occur most frequently with about one base pair per thousand base pairs in humans (see WO 00/18960).

Such variations in the genome may in many cases be associated with the occurrence of genetic diseases. Classical examples are Huntington's, cystic fibrosis, Duchenne muscular dystrophy and certain types of breast cancer (see WO 00/18960). Recently, an association between diseases such as Alzheimer's and Parkinson's and single mutations at the molecular level has been suggested.

These mutations are ordinarily single nucleotide polymorphisms (SNPs). There is thus a considerable interest in medical research in finding new positions in the genome where SNPs occur. On the other hand, the interest in investigating SNPs whose position in the genome is known exactly to the nucleotide exists primarily for the diagnosis of diseases with a molecular basis.

A number of methods for routine investigation of such SNPs at a known position in the genome have therefore been developed in the past.

Thus, miniaturized high-density oligonucleotide arrays have been produced by photolitographic synthesis. A complementary probe for every possible allele exists on these arrays. It is possible with prototypes of such chips for genotyping to investigate up to 3 000 SNPs simultaneously (Sapolsky et al, Genet. Anal., 1999, 14:187-192).

A similar method, which is likewise based on hybridization of the allele to be investigated with a complementary oligonucleotide probe, has been developed by Axys Pharmaceuticals. This method uses oligonucleotide probes which are coupled to fluorescence-labeled microspheres. These probes are hybridized directly with polymerase chain reaction (PCR) products which are likewise fluorescence-labeled. Detection then takes place in a conventional flow cytometer. It is possible in this way to investigate up to eight polymorphic genes simultaneously (Armstrong et al, Cytometry, 2 000, 40:102-108).

Whereas in these methods the hybridization takes place after a possible PCR DNA amplification step, See et al. take the opposite route. Their method uses primers with different fluorophores on the 5′ nucleotides, whose 3′ end is located at the nucleotide to be investigated. A PCR product results only with the primer which is also complementary at the 3′ end to the nucleotide to be investigated. The samples are then analyzed according to size and fluorescence by electrophoresis (See et al, Biotechniques, 2 000, 28:710-714).

A very elegant method for characterizing SNPs does not use a complete PCR, but uses only the extension of a primer by a single, fluorescence-labeled dideoxyribonucleic acid molecule (ddNTP) which is complementary to the nucleotide to be investigated. The nucleotide at the polymorphic site can be identified through detection of the primer which has been extended by one base and which is thus fluorescence-labeled (Kobayashi et al, Mol. Cell. Probes, 1995, 9:175-182). A disadvantage of this method is, however, that only a single polymorphism can be investigated in one reaction at the same time.

A possible solution to this problem is to incorporate an unambiguously identifiable sequence, which is referred to as ZipCode, into the primer. This ZipCode is recognized by a complementary ZipCode (the cZipCode) which is covalently bonded to a fluorescent microsphere. Microsphere decoding and SNP typing then takes place in a conventional flow cytometer. The ZipCode system permits analysis of a large number of SNPs with a limited number of ZipCode-coupled microspheres (Chen et al, Genome Res., 2 000, 10:549-557).

The two last-mentioned methods, which are based on extension of a primer with a fluorescence-labeled dideoxynucleotide, have a substantial advantage: fluorescence-labeled dideoxynucleotides which are optimized for high fluorescence yield and for incorporation into DNA by naturally occurring or genetically modified polymerases can be obtained at reasonable cost because they are used for the Sanger chain-termination method of DNA sequencing (Sanger et al, Proc. Nat. Acad. Sci. USA, 1977, 74:5463).

However, the two last-mentioned methods which are based on the extension of a primer with a fluorescence-labeled dideoxynucleotide are, just like the other methods, also complicated to perform.

In the method explained above without ZipCode, it is necessary in order to obtain a clear signal to prepurify the sample before application to a denaturing gel. To remove the excess of dideoxynucleotide not incorporated into the primer, it is recommended to treat the sample with alkaline phosphatase and then precipitate the primer with isopropanol. The precipitation step is particularly difficult to automate.

The ZipCode method dispenses with the intensive manual operational steps but, on the other hand, a technically elaborate flow cytometer covering a large wavelength range is necessary. In addition, there is a risk of misinterpretation of signals because the spectra of the various fluorescent dyes overlap at least in part.

In addition, when DNA from donors having two different alleles of the polymorphic DNA section to be investigated is used, there is the difficulty that the two alleles must either initially be isolated individually or selectively amplified.

It is an object of the present invention to provide methods for characterizing nucleic acid polymorphisms which do not have these prior art disadvantages.

This object is achieved by a method for characterizing nucleic acid polymorphisms, comprising the steps:

(a) provision of a nucleic acid template to be investigated,

(b) annealing of at least one starting primer onto the nucleic acid template, with the 3′ end of the primer being located upstream of a nucleic acid polymorphism to be investigated,

(c) extension of the starting primer with at least one fluorescence-labeled nucleotide and

(d) detection of nucleotides incorporated into the starting primer by single-molecule determination.

The nucleic acid polymorphism is, in the simplest case, a single nucleotide polymorphism (SNP). The polymorphism may, however, also affect a plurality of nucleotides, for example up to 20 consecutive nucleotides, or even a plurality of groups of one or more consecutive nucleotides.

It is possible to use as nucleic acid template DNA of any origin, for example from prokaryotes, in particular pathogenic prokaryotes, archaeae or eukaryotes, especially mammals, especially humans. However, it may also be recombinantly produced DNA or synthetic DNA. The DNA is preferably used in single-stranded form. Such DNA can be produced for example by reverse transcription of an RNA molecule by a reverse transcriptase, for example the AMV (avian myeloblastosis virus) or MMLV (Moloney murine leucemia virus) reverse transcriptase. However, it is also possible for double-stranded DNA, for example genomic DNA, DNA of a plasmid or of an episomal genetic element to be separated into single-stranded DNA by heating, where appropriate for one strand to be purified or enriched, and then for the primer to be annealed. The RNA or DNA is preferably a mixture of maximum homogeneity. However, since the starting primer has specificity for the DNA to be investigated, it is also possible to use heterogeneous mixtures.

The starting primer preferably consists of single-stranded DNA. However, it is of course also possible to use RNA molecules. The starting primer may also be a nucleic acid analog, for example a peptide-nucleic acid, in which case the phosphate-sugar backbone of the nucleic acids is replaced by a peptide-like backbone, for example consisting of 2-aminoethylene-glycine (Nielsen et al., Science, 254:1497-1500) as carrier of the individual bases A, T, G, C. Such a peptide-nucleic acid primer must have a 3′ end which permits elongation.

The starting primer preferably binds immediately upstream of the SNP to be characterized. However, if deoxynucleotides, and not chain-termination molecules, are employed, it is also possible to use a starting primer which binds further upstream, preferably not more than 5 nucleotides upstream from the polymorphism site to be investigated.

The fluorescence-labeled nucleotide may be both a deoxynucleotide and a chain-termination molecule. The fluorescence-labeling groups can be selected from the known fluorescence-labeling groups used for labeling biopolymers, e.g. nucleic acids, such as, for example, fluorescein, rhodamine, phycoerythrin, Cy3, Cy5 or derivatives thereof etc. The dyes can be distinguished via the wavelength, via the lifetime of the excited states or via a combination thereof.

If a plurality of nucleotides provided with different fluorescent labels are used, they can be distinguished through the wavelength of the exciting light, of the emitted light or a combination thereof. The fluorescent dyes can also be distinguished by measuring the lifetime of the excited state. It is appropriate to combine the methods. Thus, for example, four fluorescent labels can be selected for the four different bases, all of which can be excited at the same wavelength and which emit at two different wavelengths, while the lifetimes of the excited states differ for the labels having the same emission wavelength.

The primer can be extended using methods of nucleic acid chemistry known from oligonucleotide synthesis. However, the extension reaction preferably takes place with enzymatic catalysis. The polymerase is chosen depending on whether RNA or DNA is used as template. A polymerase without exonuclease activity is preferably selected. Examples of possible polymerases are T7 polymerase or thermostable polymerases such as Taq, Pfu, Pwo and the like, which are normally used for PCR reactions.

The fluorescence of a single molecule can be detected by any method of measurement, e.g. with spatially resolved or/and time-resolved fluorescence spectroscopy, which is able to pick up fluorescence signals extending down to single-photon counting in a very small volume element as present in a microchannel.

For example, detection is possible by confocal single-molecule detection such as, for example, through fluorescence correlation spectroscopy, in which case a very small, preferably a confocal volume element, for example 0.1×10⁻¹⁵ to 20×10⁻¹² l of the sample liquid flowing through the microchannel is exposed to an exciting laser light which excites the fluorescent labels present in this measurement volume to emit fluorescent light, with the emitted fluorescent light from the measurement volume being measured by means of a photodetector, and a correlation being set up between the change in the measured emission with time and the relative flow rate of the molecules involved, so that with appropriately great dilution single molecules can be identified in the measurement volume. Reference is made to details of carrying out the method and apparatus details for the devices used for the detection to the disclosure of European patent 0 679 251. Confocal single-molecule determination is moreover described by Rigler and Mets (Soc. Photo-Opt. Instrum. Eng. 1921 (1993), 239 ff.) and Mets and Rigler (J. Fluoresc. 4 (1994), 259-264).

Alternatively or additionally, the detection can also take place by time-resolved measurement of decay, called time gating, as described for example by Rigler et al., “Picosecond Single Photon Fluorescence Spectroscopy of Nucleic Acids”, in: “Ultrafast Phenomena”, D. H. Auston, ed., Springer 1984. In this case, excitation of the fluorescent molecules takes place inside the measurement volume and then—preferably after a time of ≧100 ps has elapsed—a detection interval on the photodetector is opened. It is possible in this way to keep background signals generated by Raman effects sufficiently small to allow essentially interference-free detection.

In a preferred embodiment of the method, the determination may also include measurement of a cross-correlated signal which originates from at least one nucleic acid molecule, or nucleic acid molecule complex, comprising two different labels, especially fluorescent labels, in which case a plurality of labeled nucleotides, primers or/and nucleic acid templates each with different labels can be employed. This cross-correlation determination is described, for example, by Schwille et al. (Biophys. J. 72 (1997), 1878-1886) and Rigler et al. (J. Biotechnol. 63 (1998), 97-109).

Detection of incorporated nucleotides preferably includes separation of the extended starting primer from unincorporated nucleotides.

The separation can take place for example as described in the patent application DE 100 23 423.2 on the basis of the different speed of migration of incorporated and unincorporated nucleotides in an electric field. Enrichments of about three powers of ten or more can typically be achieved in this way.

If the primer or the nucleic acid template is immobilized on a carrier particle, this particle can be trapped for example with the aid of an infrared laser. Then, a washing step can subsequently take place in a directed flow which may be electroosmotic or hydrodynamic. Hydrodynamic flow is preferred because the flow profile is more favorable and the flow rates are higher.

It is possible during the detection additionally to check whether actually incorporated nucleotides are being observed or whether free nucleotides are still present as contaminants. This is possible for example by fluorescence correlation spectroscopy. This method utilizes the fact that the extended starting primer diffuses considerably more slowly than the free chain-termination molecules and therefore remains longer in the region illuminated by a confocal microscope, so that emitted fluorescent light from the extended starting primer has a considerably longer correlation time than fluorescent light from a free chain-termination molecule. Correlators of low technical complexity are adequate for measuring the diffusion-limited correlation time, because the correlation times are in the region of ms up to a few 100 ms.

A further possibility for optically distinguishing incorporated and unincorporated chain molecules is to utilize energy-transfer processes. Thus, for example, Edman et al. (Edman, L., Mets, O. and Rigler, R., Proc. Nat. Acad. Sci. USA 93, 6710-6715 (1996)) showed that the lifetime of an excited state of tetramethyl-rhodamine is drastically shortened when the vicinity in space is large, which, with high dilution of the chain-termination molecule, occurs only when the molecule has in fact been covalently connected to the starting primer.

However, according to a further aspect of the present invention, it is also possible for the incorporated nucleotides to be digested off again, for example by an exonuclease, and to be detected singly. In this case, at least part of the sequence of the extended starting primer is determined. The methods which can be used for this are such as described in the patent application DE 100 31 840.1 and the publication of Dorre et al., Bioimaging 5, 139-152.

To carry out the sequencing reaction, the nucleic acid template or, more preferably, the starting primer is coupled to a carrier particle.

The single-molecule sequence determination preferably includes the steps:

(a) introduction of the carrier particle into a sequencing device including a microchannel,

(b) retention of the carrier particle in the sequencing device,

(c) progressive elimination of individual nucleotide units from the immobilized nucleic acid molecule,

(d) at least partial determination of the base sequence of the nucleic acid molecule on the basis of the sequence of the eliminated nucleotide units.

Detection and manipulation of loaded carrier particles can take place for example by the methods described in Holm et al. (Analytical Methods and Instrumentation, Special Issue μTAS 96, 85-87), Eigen and Rigler (Proc. Natl. Acad. Sci. USA 91 (1994), 5740-5747) or Rigler (J. Biotech. 41 (1995), 177-186), which include detection with a confocal microscope. The loaded carrier particles are manipulated in microchannel structures preferably with the aid of a trapping laser, e.g. an infrared laser. Suitable methods are described for example by Ashkin et al. (Nature 330 (1987), 24-31) and Chu (Science 253 (1991), 861-866).

The retention of the carrier particle is preferably effected by an automated process. For this purpose, the carrier particles are passed in hydrodynamic flow through the microchannel and past a detection element. The detector in the detection window is adjusted so that it recognizes a labeled sphere on the basis of the fluorescence-labeled DNA present thereon and/or an additional fluorescence-labeled probe, and then automatically brings about activation of the trapping laser in the measuring space.

An exonuclease is used to eliminate single nucleotides from the extended starting primer molecule, for example T7 DNA polymerase as exonuclease, E. coli exonuclease I or E. coli exonuclease III.

In the simplest case, only a single starting primer is employed for the extension reaction. However, it is also possible to employ and to extend a plurality of starting primers binding to different sites on the template. The starting primers then preferably have different codings, for example through different fluorescent labels or through different combinations of fluorescent labels. For identification of the starting primer it is possible in particular to incorporate fluorescence-labeled dNTPs in the starting primer. If a different fluorescent label is used for each nucleotide, it is possible with n fluorescence-labeled positions to distinguish 4ⁿ different starting primers. An even larger number results if different fluorescence-labeled analogs are employed at different positions for the same nucleotide.

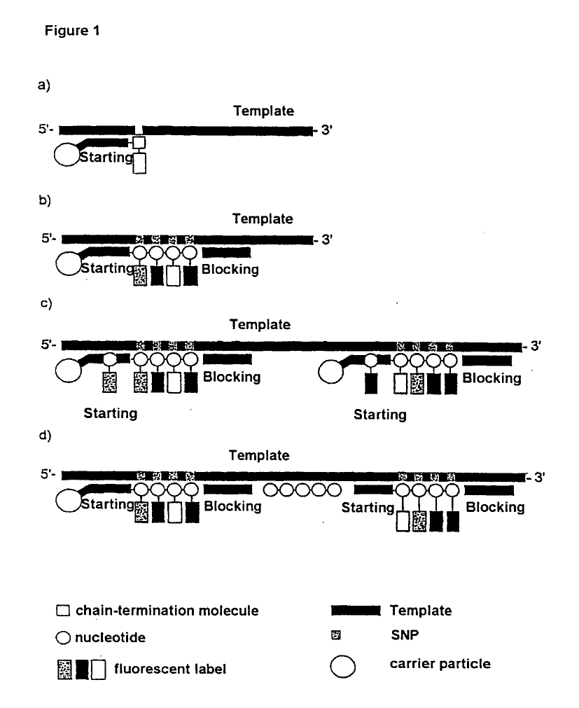

In a first embodiment, the extension reaction takes place by attaching a single, fluorescence-labeled chain-termination molecule to the starting primer(s) (see FIG. 1a for an example). Dideoxynucleotides are preferably used as chain-termination molecules. However, it is also possible to use deoxyribonucleic acids which have been modified in other ways as long as they are still recognized by the enzymes used. A conceivable example is to modify the 3′ position of the deoxyribose molecule by a halogen atom or an alkyl or alkoxy residue.

In a second embodiment of the present invention it is possible for a plurality of consecutive nucleotides to be characterized. In this case, termination of the extension reaction is induced not by incorporating a suitable chain-termination molecule but by a blocking primer (see FIG. 1b for an example). The blocking primer is bound to the nucleic acid template downstream of the polymorphism to be investigated and is itself protected against extension at its 3′ end by suitable chemical modification. For example, the nucleotide which is located furthest downstream in the blocking primer may be a chain-termination molecule. In this embodiment it is also possible to employ a plurality of starting/blocking primer pairs having different codings and able to bind to different sites on the template (see FIG. 1c for an example).

The blocking of the blocking primer may be reversible, where appropriate with the exception of the blocking of the blocking primer which binds furthest downstream. A protective group which can eliminated, for example a photolabile protective group, can be used for reversible blocking. The blocking primers particularly preferably carry at the 3′ end a phosphate group on the 3′ position of the sugar. This phosphate group at the 3′ end prevents elongation by polymerase and, for deblocking, can be eliminated directly using a 3′-phosphatase.

After the extension reaction of the starting primer there is still no covalent bond to the blocking primer which is located immediately downstream. This bond can, however, be formed for example enzymatically using a ligase. The ligation takes place considerably more easily when the blocking primers carry a phosphate group at their 5′ end.

In a third embodiment it is possible for the gap(s) between pairs composed of a starting primer extended by fluorescent nucleotides and of the blocking primer located downstream in each case, after removal of the 3′ blocking of the blocking primers, to be filled in by deoxyribonucleotides and for covalent bonds to be formed between the extended blocking primers and the starting primers located directly downstream (see FIG. 1d for an example). For this purpose, the blocking primers preferably carry a 5′-phosphate. It is not absolutely necessary in this embodiment for the various starting/blocking primer pairs to be provided with codings.

A further aspect of the invention is the combination of the chain-termination labeling with a detection in completely or partly transparent microwells (see patent application DE 100 23 421.6). This method includes the steps:

(a) provision of a carrier particle with a nucleic acid molecule which is immobilized thereon and consists of a single-stranded nucleic acid template and of a starting primer,

(b) extension of the starting primer by a fluorescence-labeled chain-termination molecule,

(c) where appropriate, washing of the well to remove unincorporated labels and

(d) detection of the fluorescent label incorporated into the starting primer.

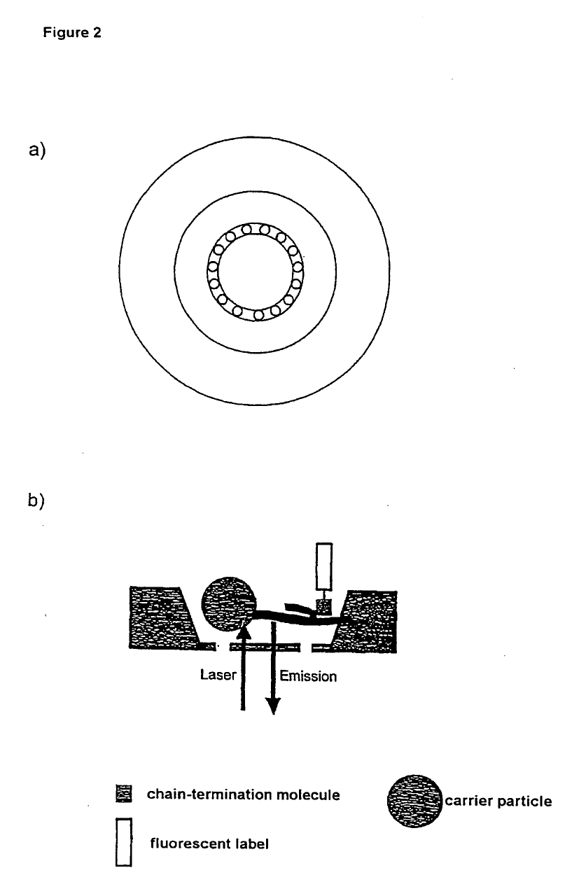

Depending on the disposition of the light source exciting the fluorescence and of the detector it is necessary to use wholly or partly transparent microwells. The excitation or/and the detection of the fluorescence can take place for example through a semiconductor laser or/and semiconductor detector integrated in the microwell (see FIG. 2 for an example). The excitation light source or/and the detector may, however, also be located outside the microstructure. The method is outstandingly suitable for automation, because a plurality of reactions can be carried out in parallel or sequentially on one microwell plate.

If the amount of starting primer and the amount of labeled nucleotide employed is kept small (nM) it is possible to distinguish incorporated and unincorporated chain-termination molecules for example by FCS (fluorescence correlation spectroscopy) as explained above. An alternative possibility, as likewise explained above, is to utilize energy-transfer processes.

A preferred alternative is to employ higher, e.g. μM, concentrations of primer and chain-termination molecules, because the incubation time can then be kept shorter. However, in this case, at least the chain-termination molecules must be removed again by a washing step after the primer extension reaction. It is possible to use for this purpose microwells having one or more small holes or a size-exclusion membrane, which retain the labeled DNA bound to a carrier particle and allow the unlabeled chain-termination molecules through (see, for example, FIG. 2).

Various combinations of starting primers and chain-termination molecules are conceivable. In the simplest case of characterization of an SNP, two or more (up to four) wells are loaded with in each case only one fluorescence-labeled chain-termination molecule and the starting primer whose 3′ end hybridizes directly in front of the nucleotide to be investigated. An elongation reaction occurs in only one of the wells. Since it is known which well contains which chain-termination molecule, the same fluorescent label can be used for all chain-termination molecules. Since the extension reaction stops when the correct nucleotide for the extension is not available, in this case it is also possible to use deoxynucleotides. It is preferred however for a chain-termination molecule as previously described, for example from the group consisting of ddATP, ddUTP, ddTTP, ddCTP and ddGTP, to be made available. A solid phase with a plurality of wells as described, for example, in the patent application DE 100 23 421.6 is preferably used. It is possible in this way to investigate a large number of SNPs in parallel in a single batch. There is preferably parallel detection of 4 wells in each case here.

However, it is also possible to employ a starting primer together with a plurality of, preferably four, different chain-termination molecules corresponding to the four nucleobases. In this case, however, the chain-termination molecules must carry different labeling groups. The labeling groups can be distinguished via the wavelength of the exciting and/or emitted light or via the lifetime of the excited state. The lifetime of the excited state is measured by measuring the fluorescence decay time (FD). In this measurement method, the molecule to be investigated is excited by a pulsed laser (e.g. a mode-locked laser). The emitted fluorescence photons are detected as a function of the time since the decay of the laser pulse, whose chronological duration must be small compared with the chronological lifetime of the excited state to be investigated.

It is possible in specific cases to use a plurality of starting primers and a plurality of chain-termination molecules in one well. For example, if it is known that only one of the bases A or T is to be expected for an SNP, and if it is known that only either G or C occur for a further SNP, it is possible to investigate the two polymorphisms in parallel. Further situations in which a plurality of nucleotide positions can be investigated simultaneously owing to additional information about the polymorphisms are readily evident to the skilled worker.

It is possible with yet another embodiment of the present invention to investigate a plurality of SNPs simultaneously even if the occurrence of all four nucleotides must be expected at the polymorphism sites. For this purpose, a starting primer whose 3′ end is located directly upstream from the nucleotide to be characterized in each case is employed for each polymorphism site. The extension reaction is then carried out with the labeled chain-termination molecules. Then, in a further step, starting primers complementary to selected restriction cleavage sites are added, so that digestion of the nucleic acid matrix to fragments of characteristic length can take place. Investigation of the diffusion characteristics of the fragments by FCS then allows the fluorescence signals to be assigned to the individual polymorphic nucleic acid positions.

A procedure which is analogous in principle is possible if a sequence-specific ligase is used in place of the restrictase. Sequence-specific ligation can be achieved for example by driving restrictases “backwards”. Since the hydrolysis reaction consumes one molecule of water, and the ligation reaction liberates one molecule of water, the equilibrium can be shifted in the direction of ligation by using a reaction medium which is as anhydrous as possible. In the analogous case of proteases, “backward operation” of the enzyme has been achieved successfully by adding large amounts of polyethylene glycol or organic solvents to the reaction buffer.

For all the embodiments described, the carrier particle preferably has a size in the range from 0.5 to 10 μm and particularly preferably from 1 to 3 μm. Examples of suitable materials for carrier particles are plastics such as polystyrene, glass, quartz, metals or metalloids such as silicon, metal oxides such as silicon dioxide or composite materials which comprise a plurality of the aforementioned components. It is particularly preferred to employ optically transparent carrier particles, for example made of plastics or particles having a plastics core and a silicon dioxide shell.

The immobilization on a carrier particle can take place either via the template or via the starting primer. In this connection, the time when the immobilization step takes place is irrelevant to the method. This step is possible i) before the hybridization step, ii) after the hybridization step, but before extension of the starting primer by the chain-termination molecule, and preferably, iii) after the extension reaction. The advantage of late immobilization is that a possible interfering effect of the carrier on the hybridization and extension reactions is avoided.

The binding of the starting primer or of the nucleic acid template to the carrier can take place by covalent or noncovalent interactions. For example, the binding of the polynucleotides to the carrier can be mediated by high-affinity interactions between the partners of a specific binding pair, e.g. biotin/streptavidin or avidin, hapten/anti-hapten antibody, sugar/lectin etc. Thus, biotinylated nucleic acid molecules can be coupled to streptavidin-coated carriers. An alternative possibility is also to bind the nucleic acid molecules to the carrier by adsorption. Thus, a binding of nucleic acid molecules which have been modified by incorporation of alkanethiol groups to metallic carriers, e.g. gold carriers, is possible. Yet a further alternative is covalent immobilization, in which case the binding of the polynucleotide can be mediated by reactive silane groups on a silica surface. If a mixture of two or more DNA molecules which differ in the site of the single nucleotide polymorphism is present as template, it is beneficial, as in the single-molecule sequencing, to bind no more than one molecule of the template or of the starting primer to a single carrier particle. This can easily be achieved by a sufficiently large molar excess of carrier particles compared with the template or the starting primer.

If, on the other hand, the DNA molecules used as template are all uniform, it is in fact beneficial, especially for the embodiment of the invention in microwells, to bind a plurality of molecules of template or starting primer to one carrier particle. Exonuclease digestion then leads to elimination of a plurality of identical fluorescence-labeled chain-termination molecules, so that the fluorescence signal and thus the signal-to-noise ratio is improved.

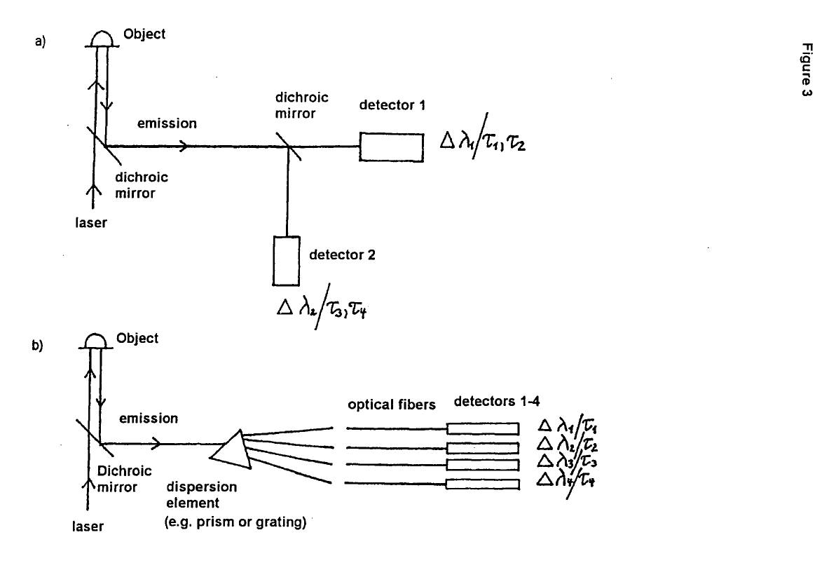

When a plurality of fluorescence-labeled components is used in the polymorphism characterizations according to the invention, the problem of effective separation of the different labels arises. As described herein before, this can take place inter alia through the use of different wavelengths for the excitation and emission of fluorescent light. The spectral splitting in this case is effected in the prior art using dichroic mirrors. Disadvantages of this procedure are the comparatively large losses, especially in the spectral splitting of the photons emitted from the fluorophore. It has surprisingly been found that the losses can be reduced if, in place of a dichroic mirror, the spectral splitting is effected with a dispersion element such as, for example, a grating, e.g. a holographic or grooved grating or a prism (see FIG. 3). It is beneficial in this case for the reflections when the light enters the dispersion element or/and when the light emerges from the dispersion element to be suppressed as completely as possible for example by suitable coating of the glass surfaces in the case of a prism. The use of a dispersion element in place of a dichroic mirror is not restricted to the use for characterizing nucleotide polymorphisms. It is likewise possible for direct detection of single molecules (see, for example, application DE 100 23 423.2), in single-molecule sequencing methods (see, for example, application DE 100 31 840.1), in methods for selecting particles (see, for example, application DE 100 31 842.8), in methods for detecting polynucleotides (see, for example, application DE 100 23 421.6), in methods for separating labeled biopolymers (see, for example, application DE 100 23 422.4) and in multiplex sequencing methods (see, for example, application DE 100 31 842.8).

FIG. 1 shows various embodiments of the polymorphism characterization. In (a) the starting primer is extended by a single fluorescence-labeled chain-termination molecule. In (b) the starting primer is extended by deoxynucleotides having different fluorescent labels up to the 3′ end of a blocking primer which binds downstream. The blocking primer itself is blocked at its 3′ end so that it is not extended. In (c), a plurality of starting/blocking primer pairs is employed. It is necessary in this case to encode the starting primers by fluorescent markers. In (d) there is likewise use of a plurality of starting/blocking primer pairs and, in addition, the blocking of the blocking primers (with the exception of the blocking of the blocking primer located furthest downstream) at the 3′ end is reversible, i.e. for example a 3′-phosphate blocking. In a first step, fluorescent nucleotides are incorporated in the presence of the 3′ blocking. After a washing step to remove unincorporated nucleotides, then, in a second step after removal of the 3′ blocking, the gap between blocking primer and following starting primer is filled in by unlabeled deoxynucleotides. The covalent bonds which are still lacking for subsequent nucleotides are formed by ligase. The result of this procedure is shown.

FIG. 2(a) shows a plan view, (b) a side view of a microwell which is suitable for use in the present invention.

FIG. 3(a) shows the optics used to date for single-molecule determination, (b) shows the optics of the invention using a dispersion element for separating the various wavelengths.

Determination can take place via the fluorescence intensities (Δλ) at various wavelengths or/and via fluorescence decay times (τ) at various wavelengths using a plurality of detectors.

1. A method for characterizing nucleic acid polymorphisms, including the steps:
(a) provision of a nucleic acid template to be investigated,
(b) annealing of at least one starting primer onto the nucleic acid template, with the 3′ end of the starting primer being located upstream of a nucleic acid polymorphism to be investigated,
(c) extension of the starting primer with at least one fluorescence-labeled nucleotide and
(d) detection of nucleotides incorporated into the starting primer by single-molecule determination.

2. The method as claimed in claim 1, characterized in that the detection of incorporated nucleotides includes a separation of the extended starting primer from unincorporated nucleotides.

3. The method as claimed in claim 1 or 2, characterized in that the detection of the incorporated nucleotide includes determination of at least part of the sequence of the extended starting primer.

4. The method as claimed in any of the preceding claims, characterized in that to carry out the single-molecule determination the starting primer is coupled to a carrier particle.

5. The method as claimed in claim 3 or 4, characterized in that the single-molecule sequence determination includes the steps:
(a) introduction of the carrier particle into a sequencing device including a microchannel,
(b) retention of the carrier particle in the sequencing device,
(c) progressive elimination of individual nucleotide units from the immobilized nucleic acid molecule,
(d) at least partial determination of the base sequence of the nucleic acid molecule on the basis of the sequence of the eliminated nucleotide units.

6. The method as claimed in any of the preceding claims, characterized in that an enzymatic elimination of nucleotides attached to the starting primer is effected by an exonuclease, in particular by T7 DNA polymerase as exonuclease, E. coli exonuclease I or E. coli exonuclease III.

7. The method as claimed in any of the preceding claims, characterized in that a single starting primer is used.

8. The method as claimed in any of claims 1 to 6, characterized in that a plurality of starting primers is used.

9. The method as claimed in either of claims 7 or 8, characterized in that the extension reaction includes the attachment of a single fluorescence-labeled chain-termination molecule.

10. The method as claimed in claim 9, characterized in that the chain-termination molecule is a dideoxynucleotide.

11. The method as claimed in either of claims 7 or 8, characterized in that the extension reaction includes the attachment of a plurality of fluorescence-labeled nucleic acid units.

12. The method as claimed in claim 11, characterized in that one or more pairs of starting and blocking primers are employed, where the 5′ end of each blocking primer binds to the nucleic acid template at a predetermined distance downstream of the 3′ end of the relevant starting primer, with the 3′ end of the blocking primer being blocked.

13. The method as claimed in claim 12, characterized in that a plurality of pairs of starting and blocking primers is employed, and the starting/blocking primer pairs can be identified by means of different codings.

14. The method as claimed in claim 12 or 13, characterized in that the blocking of the 3′ end of the blocking primer is reversible, where appropriate with the exception of the blocking primer which binds furthest downstream.

15. The method as claimed in any of claims 12 to 14, characterized in that blocking primers carrying a 3′-phosphate group are used.

16. The method as claimed in any of claims 12 to 15, characterized in that a covalent bond is formed between the starting primer extended by fluorescent nucleotides, and the blocking primer.

17. The method as claimed in any of claims 12 to 16, characterized in that the covalent bond is formed enzymatically, for example by using a ligase.

18. The method as claimed in claim 16 or 17, characterized in that at least one blocking primer carries a 5′-phosphate group.

19. The method as claimed in any of claims 14 to 18, characterized in that the gap(s) between the starting primers extended by fluorescent nucleotides, and the blocking primers located downstream in each case, after removal of the 3′ blocking of the blocking primer, are filled in by deoxyribonucleotides, and covalent bonds are formed between the extended blocking primers and the starting primers located directly downstream.

20. A method for characterizing nucleic acid polymorphisms in a microwell, including the steps:
(a) provision of a carrier particle with a nucleic acid molecule which is immobilized thereon and consists of a single-stranded nucleic acid template and of a starting primer,
(b) extension of the starting primer by a fluorescence-labeled chain-termination molecule,
(c) where appropriate washing of the well to remove unincorporated labels and
(d) detection of the fluorescent label incorporated into the starting primer.

21. The method as claimed in claim 20, characterized in that a semiconductor laser or/and semiconductor detector integrated into the microwell is used for the excitation or/and the detection of the fluorescence.

22. The method as claimed in claim 20 or 21, characterized in that a plurality of reactions is carried out in parallel or sequentially on one microwell plate.

23. The method as claimed in any of claims 20 to 22, characterized in that only one type of labeled nucleotide selected from the group consisting of ddATP, ddUTP, ddTTP, ddCTP, ddGTP is available for the extension of the starting primer.

24. The method as claimed in any of claims 20 to 22, characterized in that a plurality of chain-termination molecules which can be distinguished by their fluorescent label is available for the extension of the starting primer.

25. The method as claimed in any of the preceding claims, characterized in that a carrier particle made of plastic, glass, quartz, metals, metalloids, metal oxides or of a composite material is used.

26. The method as claimed in any of the preceding claims, characterized in that the carrier particle has a diameter of from 1 nm to 10 μm.

27. The method as claimed in any of the preceding claims, characterized in that the the nucleic acid matrix is immobilized on the carrier particle via a 5′ terminus or the starting primer is immobilized via its 3′ terminus by means of bioaffinity interactions.

28. The method as claimed in claim 1, characterized in that a biotinylated nucleic acid molecule is immobilized on an avidin- or streptavidin-coated carrier particle.

29. The method as claimed in any of the preceding claims, characterized in that the different fluorescent labels are distinguished on the basis of the wavelength, the lifetime of the excited state or a combination thereof.

30. The method as claimed in any of the preceding claims, characterized in that the determination takes place by confocal single-molecule detection or/and by time-resolved decay measurement.

31. The method as claimed in any of the preceding claims, characterized in that the determination includes the measurement of a cross-correlated signal which originates from a nucleic acid molecule, or nucleic acid molecule complex, comprising at least two different labels, especially fluorescent labels.

32. A method for increasing the detection efficiency in the detection of the fluorescence from single molecules, characterized in that a dispersion element is used to separate the light of different wavelengths.

33. The method as claimed in claim 32, characterized in that a prism or grating is used as dispersion element.
